(19) United States
(12) Patent Application Publication
(10) Pub. No.: US 2020/0194128 A1
(43) Pub. Date: Jun. 18, 2020

(54) Brain Simulator

(71) Applicant: Du Cheng, New York, NY (US)

(72) Inventor: Du Cheng, New York, NY (US)

(21) Appl. No.: 15/904,775

(22) Filed: Feb. 26, 2018

(51) Int. Cl. G16H 50/50

(52) U.S. Cl. CPC G16H 50/50

(57) ABSTRACT

This invention relates to an apparatus for the creation of an anatomical model from an anatomical scan. The invention analyses an anatomical scan of a patient and then creates an anatomical model of that scan that is representative of the actual human head. In other uses the model can be constructed from multiple sample patients to create a model that is an example patient for a type of condition or disease which is desired to be modeled and studied. The invention manipulates the threshold of the CT scan to use the airspace around the tissue to construct a mold. In this way the airspace that surrounds the skull is first printed. Then the holes are sealed on the 3D printed skull and dropped into the mold. After this step is done you are then able to pour fluidic rubber materials in the gap between the model and the mold. Developing the mold in this manner enables the ability to rapidly re-produce a multi-tissue layered model with accurate anatomy of both soft tissue and bony structure. After the basic structure of the model is made other structures such as eyes and muscles can be made and attached to the skull before dropping into the mold sometimes, and then all the structures are embedded into the soft tissue/skin. In addition, the system and methods allows for other structures such as a tumor to be embedded as well.

### CROSS REFERENCE TO RELATED APPLICATIONS

Not applicable.

### REFERENCE TO GOVERNMENT FUNDING SOURCES

Not applicable.

### REFERENCE TO SEQUENCE LISTING

Not applicable.

### FIELDS OF THE INVENTION

The disclosure as detailed herein is in the technical field of.

### DESCRIPTION OF RELATED ART

### General Summary of the Invention

The invention relates to a system and method to make a surgical training model that has bone/soft tissue that are realistically constructed based on a CT scan of a patient. So in this way the models can be patient-specific. In other uses the model can be constructed from multiple sample patients to create a model that is an example patient for a type of condition or disease which is desired to be modeled and studied. The invention manipulates the threshold of the CT scan to use the airspace around the tissue to construct a mold. In this way the airspace that surrounds the skull is first printed. Then the holes are sealed on the 3D printed skull and dropped into the mold. After this step is done you are then able to pour fluidic rubber materials in the gap between the model and the mold. Developing the mold in this manner enables the ability to rapidly re-produce a multi-tissue layered model with accurate anatomy of both soft tissue and bony structure. After the basic structure of the model is made other structures such as eyes and muscles can be made and attached to the skull before dropping into the mold sometimes, and then all the structures are embedded into the soft tissue/skin. In addition, the system and methods allows for other structures such as a tumor to be embedded as well.

Once constructed the models can be used for realistic hands-on surgical training for example craniosynostosis. It is also contemplated that the models could also be used for ballistic analyses.

### DESCRIPTION OF FIGURES

FIG. 1 is a perspective view, which shows a diagram view which shows an exemplary hardware architecture of a computing device used in an embodiment of the invention.

FIG. 2 is a perspective view, which shows a diagram view which shows an exemplary logical architecture for a client device, according to an embodiment of the invention.

FIG. 3 is a perspective view, which shows a diagram of an exemplary architectural arrangement of clients, servers, and external services, according to an embodiment of the invention.

FIG. 4 is a perspective view, which shows a diagram view which shows an embodiment of a hardware architecture of a computing device used in various embodiments of the invention.

FIG. 5 is a perspective view, which shows an overview of the system.

FIG. 6 is a perspective view, which shows an anatomical model.

FIG. 7 is a perspective view, which shows ventral view of an anatomical model.

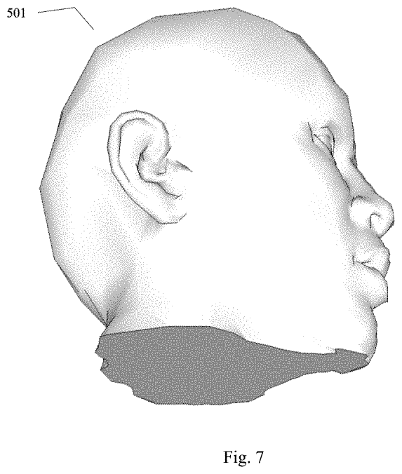

FIG. 8 is a perspective view, which shows a 3d printed skull.

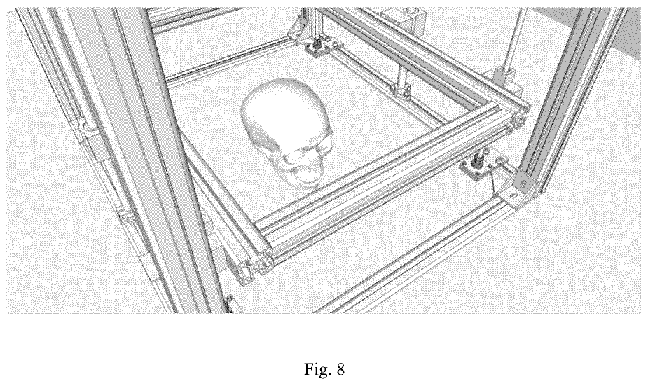

FIG. 9 is a perspective view, which shows a close up of a 3D printed skull.

FIG. 10 is a perspective view, which shows spacers on top of the skull.

FIG. 11 is a perspective view, which shows mold bottom.

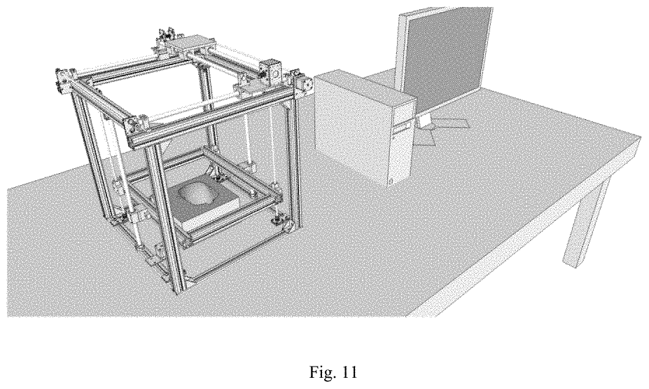

FIG. 12 is a perspective view, which shows mold top.

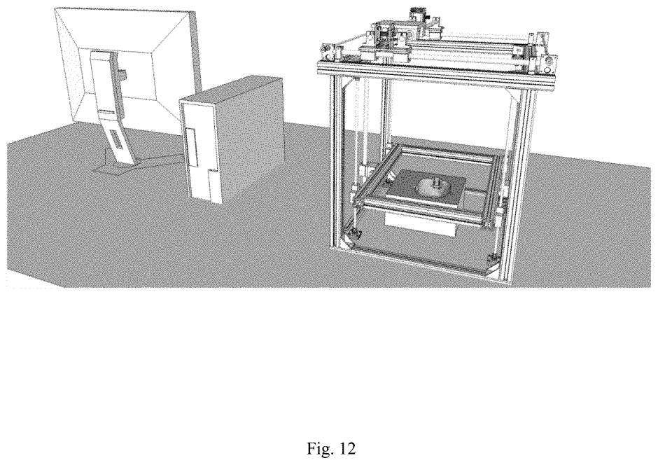

FIG. 13 is a perspective view, which shows creation of the mold prior to pouring.

FIG. 14 is a perspective view, which shows an alternate view of the creation of the mold prior to pouring.

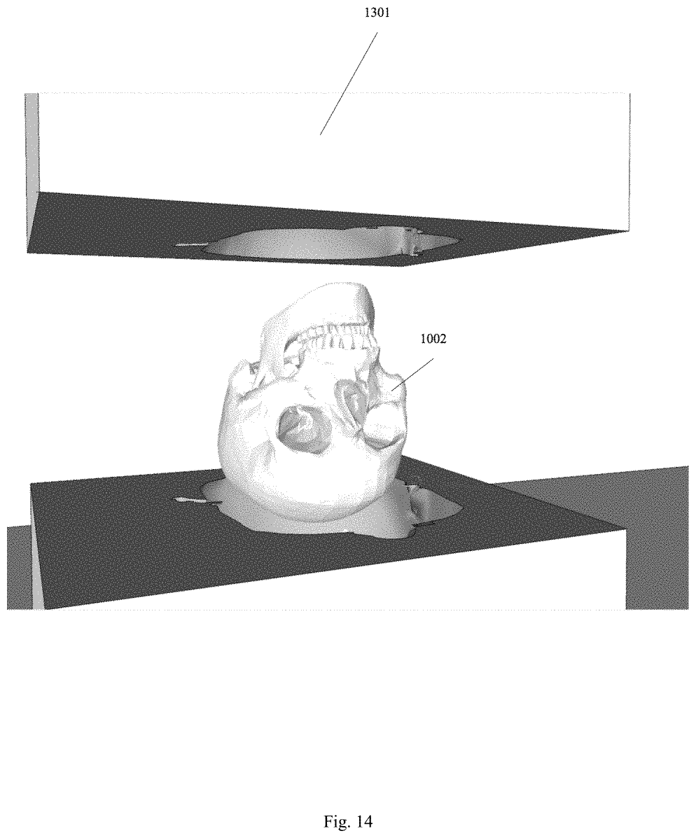

FIG. 15 is a perspective view, which shows a closed mold.

FIG. 16 is a perspective view, which shows the extracted anatomical model.

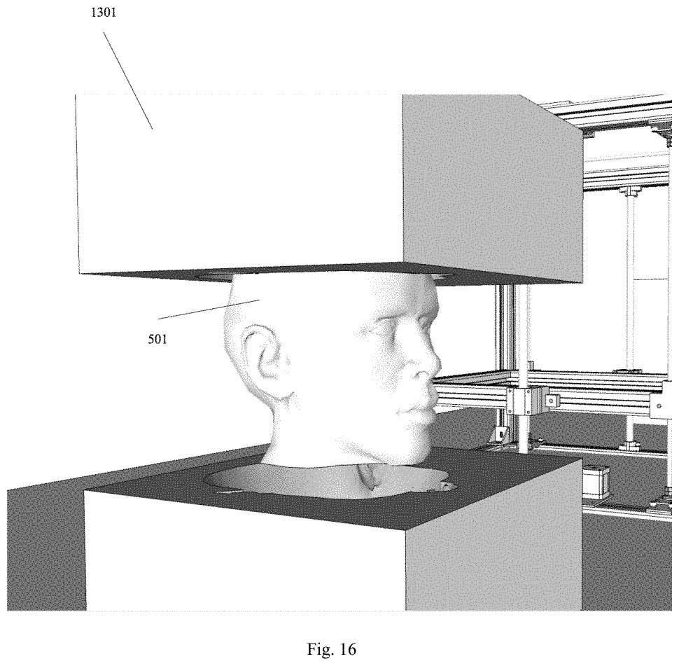

FIG. 17 is a perspective view which shows an internal model

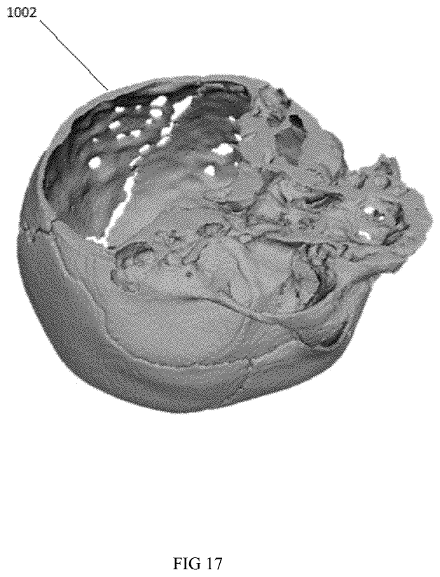

FIG. 18 is a perspective view which shows a mold model

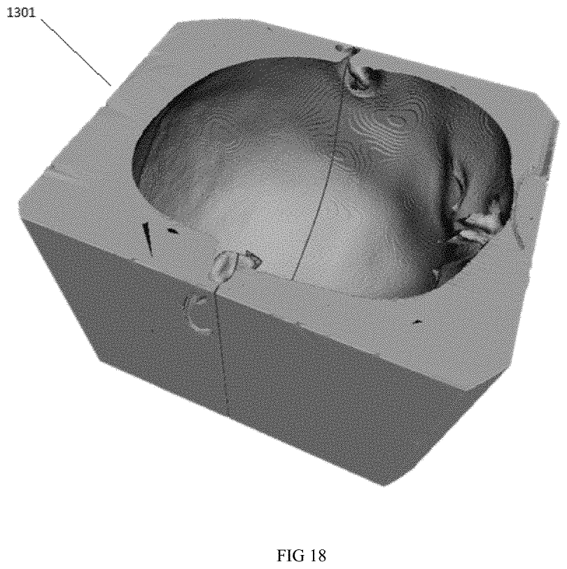

FIG. 19 is a perspective view which shows an internal model, a mold model and an internal mold model pair

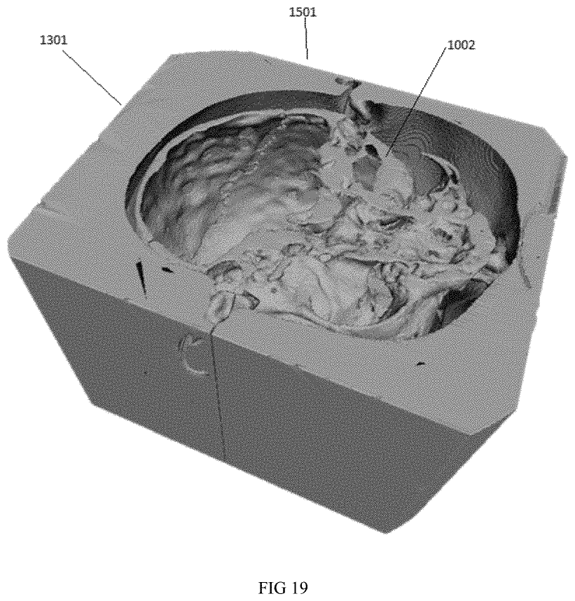

FIG. 20 is a perspective view which shows one half of a mold model

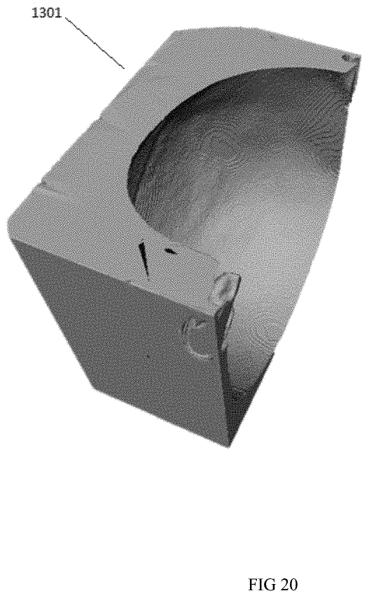

FIG. 21 is a perspective view which shows one half of a mold model and an internal model

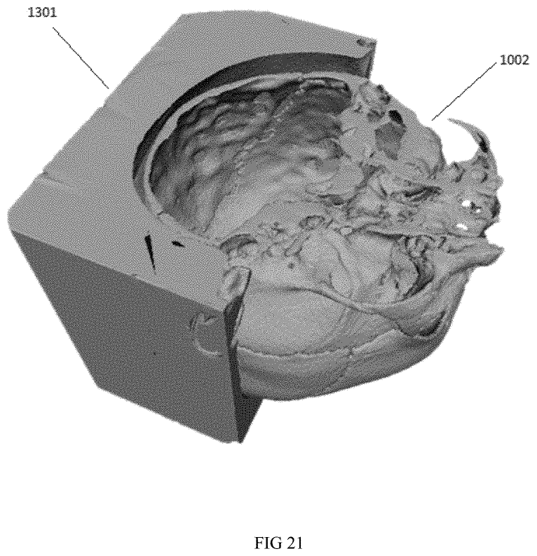

FIG. 22 is a diagram, which shows overall use of system.

FIG. 23 is a diagram, which shows Operable Anatomical Model.

FIG. 24 is a diagram, which shows the operable model threshold parameters.

FIG. 25 is a diagram of Creation of soft tissue creator material.

FIG. 26 is a diagram of the Creation of the internal model.

FIG. 27 is a diagram of the Creation of the mold.

FIG. 28 is a diagram of Generation of mold model file.

FIG. 29 is a diagram of Generation of model for the internal structure.

FIG. 30 is a diagram of Creation of internal model mold model pair.

FIG. 31 is a diagram of Cropping of anatomical scan to the mold ROI.

FIG. 32 is a diagram of Selection of the ROI.

FIG. 33 is a diagram of Loading an anatomical scan.

FIG. 34 is a diagram of Creation of brain simulator.

FIG. 35 is a diagram of Generation of model for the internal structure.

### DETAILED DESCRIPTION

One or more different inventions may be described in the present application. Further, for one or more of the inventions described herein, numerous alternative embodiments may be described; it should be appreciated that these are presented for illustrative purposes only and are not limiting of the inventions contained herein or the claims presented herein in any way. One or more of the inventions may be widely applicable to numerous embodiments, as may be readily apparent from the disclosure. In general, embodiments are described in sufficient detail to enable those skilled in the art to practice one or more of the inventions, and it should be appreciated that other embodiments may be utilized and that structural, logical, software, electrical and other changes may be made without departing from the scope of the particular inventions. Accordingly, one skilled in the art will recognize that one or more of the inventions may be practiced with various modifications and alterations. Particular features of one or more of the inventions described herein may be described with reference to one or more particular embodiments or figures that form a part of the present disclosure, and in which are shown, by way of illustration, specific embodiments of one or more of the inventions. It should be appreciated, however, that such features are not limited to usage in the one or more particular embodiments or figures with reference to which they are described. The present disclosure is neither a literal description of all embodiments of one or more of the inventions nor a listing of features of one or more of the inventions that must be present in all embodiments.

Headings of sections provided in this patent application and the title of this patent application are for convenience only, and are not to be taken as limiting the disclosure in any way.

Devices that are in communication with each other need not be in continuous communication with each other, unless expressly specified otherwise. In addition, devices that are in communication with each other may communicate directly or indirectly through one or more communication means or intermediaries, logical or physical.

A description of an embodiment with several components in communication with each other does not imply that all such components are required. To the contrary, a variety of optional components may be described to illustrate a wide variety of possible embodiments of one or more of the inventions and in order to more fully illustrate one or more aspects of the inventions. Similarly, although process steps, method steps, algorithms or the like may be described in a sequential order, such processes, methods and algorithms may generally be configured to work in alternate orders, unless specifically stated to the contrary. In other words, any sequence or order of steps that may be described in this patent application does not, in and of itself, indicate a requirement that the steps be performed in that order. The steps of described processes may be performed in any order practical. Further, some steps may be performed simultaneously despite being described or implied as occurring non-simultaneously (e.g., because one step is described after the other step). Moreover, the illustration of a process by its depiction in a drawing does not imply that the illustrated process is exclusive of other variations and modifications thereto, does not imply that the illustrated process or any of its steps are necessary to one or more of the invention(s), and does not imply that the illustrated process is preferred. Also, steps are generally described once per embodiment, but this does not mean they must occur once, or that they may only occur once each time a process, method, or algorithm is carried out or executed. Some steps may be omitted in some embodiments or some occurrences, or some steps may be executed more than once in a given embodiment or occurrence.

When a single device or article is described herein, it will be readily apparent that more than one device or article may be used in place of a single device or article. Similarly, where more than one device or article is described herein, it will be readily apparent that a single device or article may be used in place of the more than one device or article.

The functionality or the features of a device may be alternatively embodied by one or more other devices that are not explicitly described as having such functionality or features. Thus, other embodiments of one or more of the inventions need not include the device itself.

Techniques and mechanisms described or referenced herein will sometimes be described in singular form for clarity. However, it should be appreciated that particular embodiments may include multiple iterations of a technique or multiple instantiations of a mechanism unless noted otherwise. Process descriptions or blocks in figures should be understood as representing modules, segments, or portions of code which include one or more executable instructions for implementing specific logical functions or steps in the process. Alternate implementations are included within the scope of embodiments of the present invention in which, for example, functions may be executed out of order from that shown or discussed, including substantially concurrently or in reverse order, depending on the functionality involved, as would be understood by those having ordinary skill in the art.

Software/hardware hybrid implementations of at least some of the embodiments disclosed herein may be implemented on a programmable network-resident machine (which should be understood to include intermittently connected network-aware machines) selectively activated or reconfigured by a computer program stored in memory. Such network devices may have multiple network interfaces that may be configured or designed to utilize different types of network communication protocols. A general architecture for some of these machines may be described herein in order to illustrate one or more exemplary means by which a given unit of functionality may be implemented. According to specific embodiments, at least some of the features or functionalities of the various embodiments disclosed herein may be implemented on one or more general-purpose computers associated with one or more networks, such as for example an end-user computer system, a client computer, a network server or other server system, a mobile computing device (e.g., tablet computing device, mobile phone, smartphone, laptop, or other appropriate computing device), a consumer electronic device, a music player, or any other suitable electronic device, router, switch, or other suitable device, or any combination thereof. In at least some embodiments, at least some of the features or functionalities of the various embodiments disclosed herein may be implemented in one or more virtualized computing environments (e.g., network computing clouds, virtual machines hosted on one or more physical computing machines, or other appropriate virtual environments).

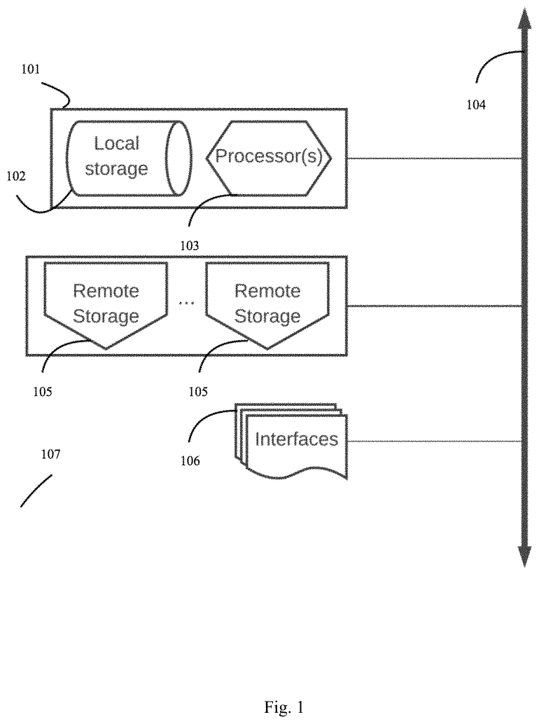

Referring now to FIG. 1, which shows a diagram view which shows an exemplary hardware architecture of a computing device used in an embodiment of the invention.

Cpu 101 comprises a unit responsible for implementing specific functions associated with the functions of specifically configured computing device or machine. The central processing unit is an acronym which stands for cpu 101. In some embodiments, it is thought that examples of cpu 101 may include: system-on-a-chip (SOC) type hardware, Qualcomm SNAPDRAGON™, or Samsung EXYNOS™ CPU.

Local memory 102 comprises one or more physical devices used to store programs (sequences of instructions) or data (e g. program state information) on a temporary or permanent basis for use in a computer or other digital electronic device, which may be configured to couple to the system in many different configurations. In some embodiments, it is thought that examples of local memory 102 may include: non-volatile random access memory (RAM), read-only memory (ROM), or one or more levels of cached memory. Local memory 102 functions to both 1) cache and/or store data and to 2) store programming instructions.

In some embodiments, it is thought that examples of processor 103 may include: Intel processor, ARM processor, Qualcomm processor, AMD processor, application-specific integrated circuits (ASICs), electrically erasable programmable read-only memories (EEPROMs), field-programmable gate arrays (FPGAs), mobile processor, microprocessor, microcontroller, microcomputer, programmable logic controller, or programmable circuit.

Communications network 104 comprises a communications network that allows computers to exchange data using known protocols. In some embodiments, it is thought that examples of communications network 104 may include: personal area network, wireless personal area network, near-me area network, local area network, wireless local area network, wireless mesh network, wireless metropolitan area network, wireless wide area network, cellular network, home area network, storage area network, campus area network, backbone area network, metropolitan area network, wide area network, enterprise private network, virtual private network, intranet, extranet, Internetwork, Internet, near field communications, mobile telephone network, CDMA network, GSM cellular networks, or WiFi network.

Remote memory 105 comprises a service that provides users with a system for the backup, storage, and recovery of data.

Interface 106 comprises a mechanism to control the sending and receiving of data packets over a computer network or support peripherals used with the computing device 107. In some embodiments, it is thought that examples of interface 106 may include: network interface cards (NICs), ethernet interfaces, frame relay interfaces, cable interfaces, DSL interfaces, token ring interfaces, graphics interfaces, universal serial bus (USB) interfaces, Serial port interfaces, Ethernet interfaces, FIREWIRE™ interfaces, THUNDERBOLT™ interfaces, PCI interfaces, parallel interfaces, radio frequency (RF) interfaces, BLUETOOTH™ interfaces, near-field communications interfaces, 802.11 (WiFi) interfaces, frame relay interfaces, TCP/IP interfaces, ISDN interfaces, fast Ethernet interfaces, Gigabit Ethernet interfaces, Serial ATA (SATA) or external SATA (ESATA) interfaces, high-definition multimedia interface (HDMI), digital visual interface (DVI), analog or digital audio interfaces, asynchronous transfer mode (ATM) interfaces, high-speed serial interface (HSSI) interfaces, Point of Sale (POS) interfaces, or fiber data distributed interfaces (FDDIs).

Computing device 107 comprises an electronic device capable of executing software- or hardware-based instructions according to one or more programs stored in memory. In some embodiments, it is thought that examples of computing device 107 may include: desktop computers, carputers, game consoles, laptops, notebooks, palmtop, tablet, smartphones, smartbooks, or a server system utilizing cpu 101, local memory 102 and/or remote memory 105, and interface 106.

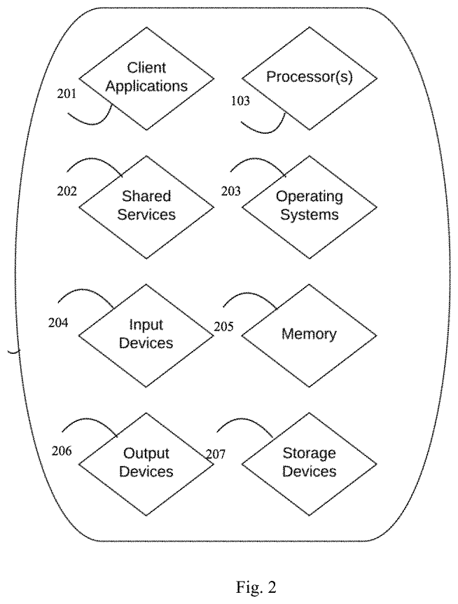

Referring now to FIG. 2, which shows a diagram view which shows an exemplary logical architecture for a client device, according to an embodiment of the invention.

Shared service 202 comprises web-enabled services or functionality related to a computing device 107.

Operating systems 203 comprises system software that manages computer hardware and software resources and provides common services for computer programs. In some embodiments, it is thought that examples of operating systems 203 may include: Microsoft's WINDOWS™, Apple's Mac OS/X, iOS operating systems, Linux operating system, or Google's ANDROID™ operating system.

Input devices 204 comprises device of any type suitable for receiving user input. In some embodiments, it is thought that examples of input devices 204 may include: keyboard, touchscreen, microphone, mouse, touchpad, or trackball.

Memory 205 comprises mechanism designed to store program instructions, state information, and the like for performing various operations described herein, may be storage devices 207, in some embodiments. In some embodiments, it is thought that examples of memory 205 may include: read-only memory (ROM), read-only memory (ROM) devices, memristor memory, random access memory (RAM), or RAM hardware modules.

Output devices 206 comprises device of any type suitable for outputing computing device 107 related information. In some embodiments, it is thought that examples of output devices 206 may include: screens for visual output, speakers, or printers.

Storage devices 207 comprises mechanism designed to store information which in some embodiments may be memory 205. In some embodiments, it is thought that examples of storage devices 207 may include: magnetic media, hard disks, floppy disks, magnetic tape, optical media, CD-ROM disks, magneto-optical media, optical disks, flash memory, solid state drives (SSD), “hybrid SSD” storage drives, swappable flash memory modules, thumb drives, thumb drives, removable optical storage discs, or electrical storage device.

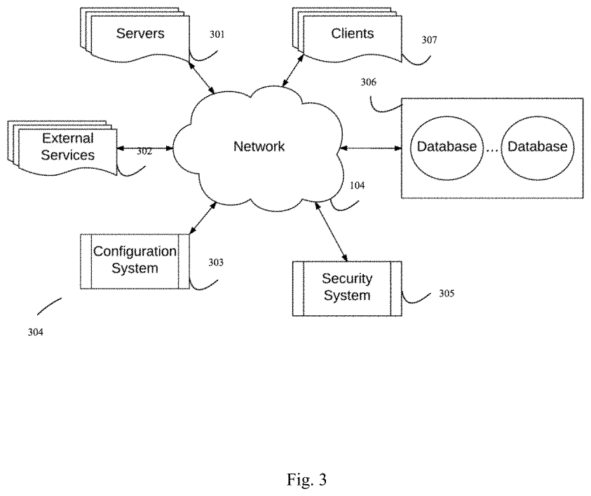

Referring now to FIG. 3, which shows a diagram of an exemplary architectural arrangement of clients, servers, and external services, according to an embodiment of the invention.

Server 301 comprises a computing device 107 configured to handle requests received from one or more client 307 over a communications network.

External service 302 comprises web-enabled services or functionality related to or installed on a computing device 107 itself which may be deployed on one or more of a particular enterprise's or user's premises.

Configuration system 303 comprises a system common to information technology (IT) and web functions that implements configurations or management system. Distributed computing network 304 comprises any number of Client and/or Server operably connected to a Communications Network for the purposes of implementing the system.

Security system 305 comprises a system common to information technology (IT) and web functions that implements security related functions for the system.

Database 306 comprises an organized collection of data within a program instructions related system, designed to allow the definition, creation, querying, update, and administration of databases. In some embodiments, it is thought that examples of database 306 may include: relational database system, NoSQL system, Hadoop system, Cassandra system, Google BigTable, column-oriented databases, in-memory databases, or clustered databases.

Client 307 comprises one or more computing device 107 with program instructions for implementing client-side portions of the present system which in some embodiments, may be connected to a communications network.

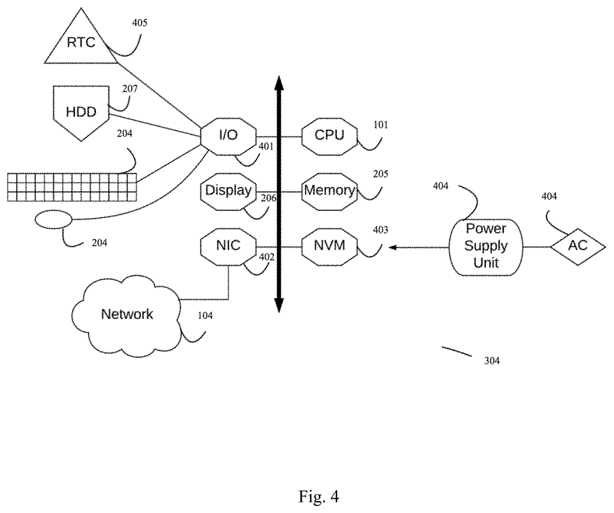

Referring now to FIG. 4, which shows a diagram view which shows an embodiment of a hardware architecture of a computing device used in various embodiments of the invention.

Input output units 401 comprises devices used by a human (or other system) to communicate with a computer.

Nic 402 comprises a computer hardware component that connects a computer to a computer network.

Non volatile memory 403 comprises computer memory that can retrieve stored information even after having been power cycled (turned off and back on).

Power supply 404 comprises an electronic device that supplies electric energy to an electrical load.

Real time clock 405 comprises a computing device 107 clock (most often in the form of an integrated circuit) that keeps track of the current time.

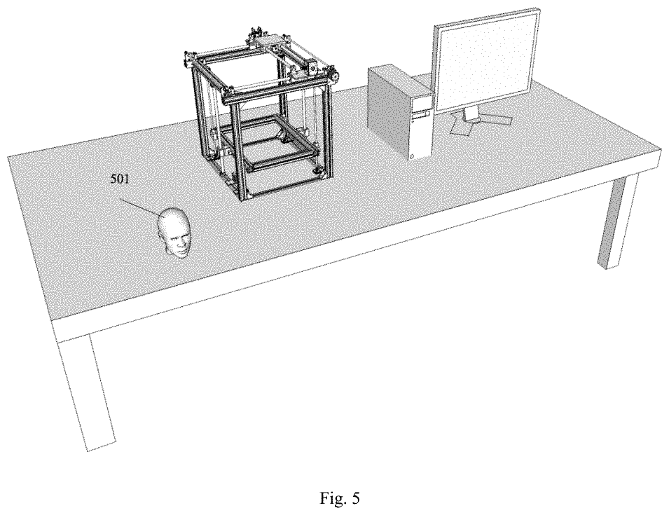

Referring now to FIG. 5, which shows an overview of system.

Operable anatomical model 501 comprises an apparatus that is anatomically identically to the original model that was the subject of an anatomical scan. Operable anatomical model 501 preferably comprises additional anatomical structure 1805, printable interior model 1804, printable mold model 1801, anatomical model 1802, and finally anatomical scenario 1803.

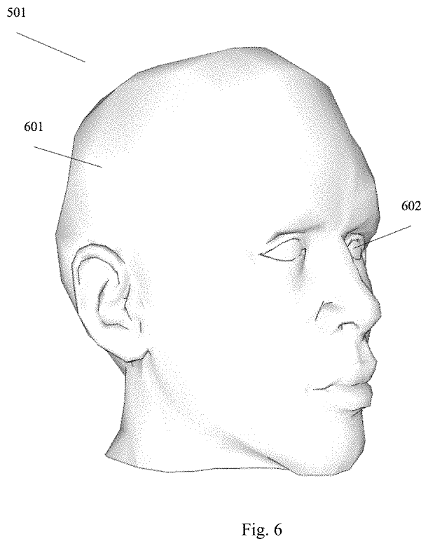

Referring now to FIG. 6, which shows an anatomical model.

External structure 601 comprises the external and less dense structure of an anatomical model 1802. Eye structure 602 comprises the simulated anatomical structure that defines the eye structure.

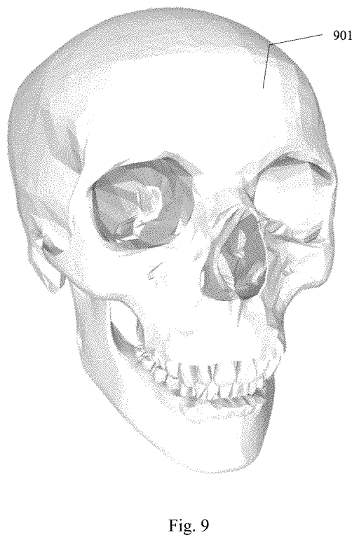

Referring now to FIG. 9, which shows close up of 3d printed skull.

Internal structure model 901 comprises the full internal model made from the operable model threshold parameters 1901 without any spacer 1001 spacers placed in.

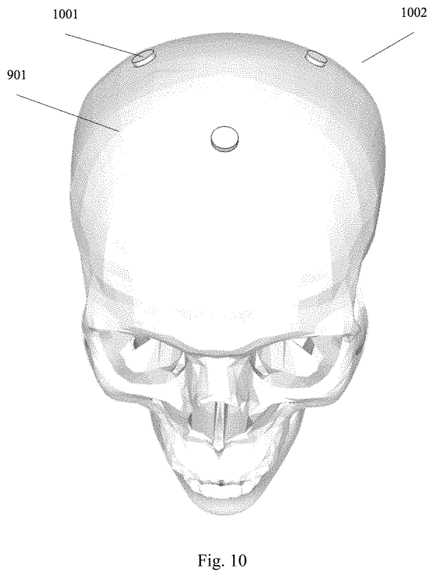

Referring now to FIG. 10, which shows spacers on top of the skull.

Spacer 1001 comprises material paced on the edge of the internal model 1002 for stability and stabilization of the internal model 1002. In some embodiments, spacer 1001 has a preferred count of 3 each but in some embodiments, may range from a minimum of 1 each to a maximum count of 100 each.

Internal model 1002 comprises the model which represents the more dense structure threshold 1906 obtained from the anatomical scan. Internal model 1002 comprises spacer receiver 1716 and internal structure model 901.

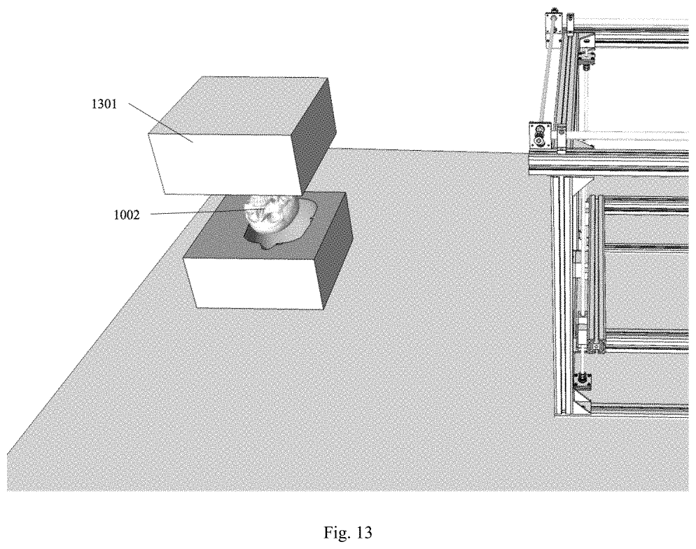

Referring now to FIG. 13, which shows creation of the mold prior to pouring.

Mold model 1301 comprises a model which has molds and defines the outer surface of the external structure 601 from an anatomical scan. Mold model 1301 is mainly thought to be composed of smooth material. Mold model 1301 preferably comprises spacer 1001.

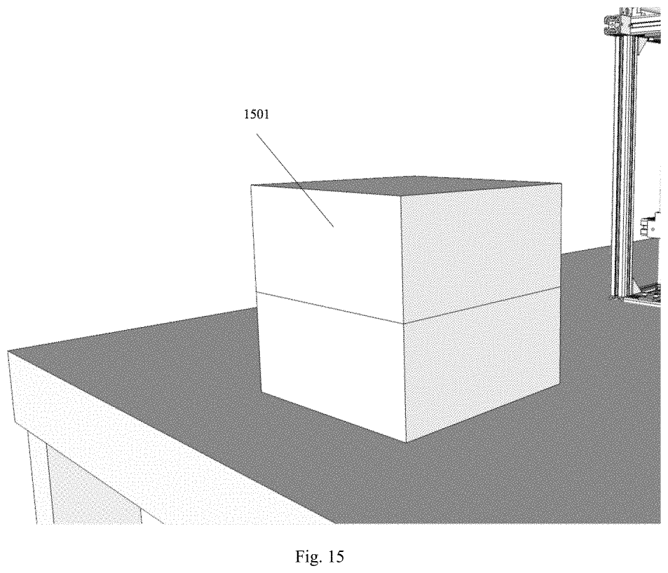

Referring now to FIG. 15, which shows closed mold.

Internal model mold model pair 1501 comprises the combined structure with matching internal model 1002 and mold model 1301 which when combined allows for the creation of the external structure 601. Internal model mold model pair 1501 comprises soft tissue creator model 1718 and soft tissue creator material 1717.

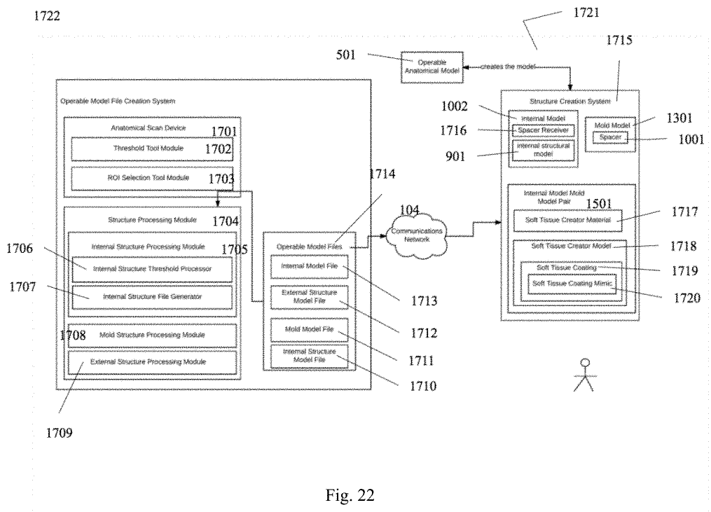

Referring now to FIG. 22, which shows overall use of system.

Anatomical scan device 1701 comprises a device or apparatus which is capable of creating an anatomical scan. In some embodiments, it is thought that if anatomical scan device 1701 is absent then a person creates an anatomical scan from the scan of a person or other object that is desired to be modeled by the operable model file creation system 1722. Anatomical scan device 1701 comprises threshold tool module 1702 and roi selection tool module 1703. Typically the threshold and ROI tool is not in the scan device. The scan device generates the raw scan and usually the post-processing software sets the threshold of generating model and cropping the ROI.

Threshold tool module 1702 comprises module which analyses an anatomical scan and determines regions which are within the operable model threshold parameters 1901. One goal of threshold tool module 1702 is to enables the operable model file creation system 1722 to determine and select certain regions on an anatomical scan.

Roi selection tool module 1703 comprises module which permits the selection of operable model threshold parameters 1901 within a roi.

Structure processing module 1704 comprises one or more modules which send receive operable model files 1714 and process and create operable model files 1714. Structure processing module 1704 preferably comprises internal structure processing module 1705, external structure processing module 1709, and finally mold structure processing module 1708.

Internal structure processing module 1705 comprises one or more modules which creates the internal model file 1713 from the anatomical scan and the more dense structure threshold 1906. Internal structure processing module 1705 comprises internal structure threshold processor 1706 and internal structure file generator 1707.

Internal structure threshold processor 1706 comprises module which processes the more dense structure threshold 1906.

Internal structure file generator 1707 comprises module which creates the internal structure model file 1710.

Mold structure processing module 1708 comprises one or more modules which accepts operable model files 1714 and creates a mold model file 1711. One goal of mold structure processing module 1708 is to enables the operable model file creation system 1722 to create one or more mold model file 1711 for creating either internal model 1002 or external structure 601 from either the internal structure model file 1710, the internal model file 1713 or the external structure model file 1712.

External structure processing module 1709 comprises one or more modules which creates external structure model file 1712 from the less dense structure threshold 1903.

Internal structure model file 1710 comprises data or data object which stores information for the creation of the internal structure by the structure processing module 1704.

Mold model file 1711 comprises data or data object which stores information needed to create the mold model 1301 by the structure processing module 1704.

External structure model file 1712 comprises data or data object which stores information for the creation of the external structure 601 by the structure processing module 1704.

Internal model file 1713 comprises data or data object which stores information outlining the parameters for construction of the internal model by the structure processing module 1704.

Operable model files 1714 comprises data or data object which stores the information needed to make the mold model 1301 by the structure creation system 1715. Operable model files 1714 preferably comprises internal model file 1713, external structure model file 1712, mold model file 1711, and finally internal structure model file 1710.

Structure creation system 1715 comprises method or system for the creation of an operable anatomical model 501. Structure creation system 1715 preferably comprises internal model 1002, internal model mold model pair 1501, and finally mold model 1301.

Spacer receiver 1716 comprises one or more indentations or apertures on the internal structure model 901 where one or more spacer 1001 can be placed. One goal of spacer receiver 1716 is to enables the internal model 1002 to receive spacer 1001.

Soft tissue creator material 1717 comprises material which is used to create the external structure 601 of a model. In some embodiments, it is thought that examples of soft tissue creator material 1717 may include: rubber, silicone rubber, urethane rubber, plastics, viral rubber, poly sulfite (elastic polymers), or elastic polymers.

Soft tissue creator model 1718 comprises a flexible polymer used to create the external structure 601. In some embodiments, it is thought that an example of soft tissue creator model 1718 could be urethane rubber or perhaps silicone rubber and the like. Soft tissue creator model 1718 preferably comprises soft tissue coating 1719.

Soft tissue coating 1719 comprises coating which is applied to the exterior of the external structure 601. In some embodiments, soft tissue coating 1719 has a preferred thickness of 0.5 millimeters but in some embodiments, may range from a minimum of 0.05 millimeters to a maximum thickness of 2 millimeters. Soft tissue coating 1719 preferably comprises soft tissue coating mimic 1720.

Soft tissue coating mimic 1720 comprises the materials used for creating the thin layer of Dura mimics in the inner surface of the skull. The soft tissue coating mimic 1720 is usually brushed onto the inner surface of the 3D printed skull with a brush and then it is left to be cured. The soft tissue coating mimic 1720 serves two purposes. 1. To seal off the cracks between the bone plates so when silicone rubbers are poured between the bone and the mold, it won't leak into the brain space. 2. To make the thin layer of rubber that mimic the dura that lines the inner surface of the skull surrounding the brain. During surgery one would need to preserve the dura structure because if the dura is punctured it risks damaging the brain and arachnid membrane of the brain which would cause bleeding.

Mold creation system 1721 comprises method, system or apparatus for the creation of an anatomical model 1802. In some embodiments, it is thought that examples of mold creation system 1721 may include: sculpting, manufacturing, CNC machining, or 3D printing. Mold creation system 1721 comprises structure creation system 1715 and operable anatomical model 501.

Operable model file creation system 1722 comprises a system of devices, interfaces, and communications that allows one to create an operable anatomical model 501 from an anatomical scan. Operable model file creation system 1722 preferably comprises anatomical scan device 1701, operable model files 1714, operable model threshold parameters 1901, mold creation system 1721, and finally structure processing module 1704.

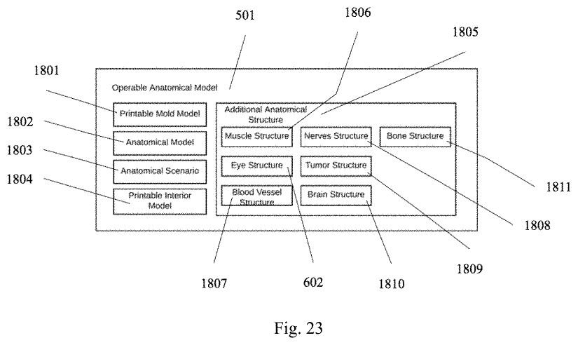

Referring now to FIG. 23, which shows Operable Anatomical Model.

Printable mold model 1801 comprises data or data object that defines a printable structure for creation of a mold.

Additional anatomical structure 1805 comprises one or more simulated anatomical structure added to the internal model 1002, designed to create an anatomical scenario 1803 wherein one or more persons engage with the operable anatomical model 1802 in order to role play an anatomical scenario 1803.

Additional anatomical structure 1805 preferably comprises blood vessel structure 1807, nerves structure 1808, tumor structure 1809, bone structure 1811, brain structure 1810, eye structure 602, and finally muscle structure 1806.

Muscle structure 1806 comprises the simulated anatomical structure that defines the muscle structure.

Blood vessel structure 1807 comprises the simulated anatomical structure that defines the blood vessel structure.

Nerves structure 1808 comprises the simulated anatomical structure that defines the nerve structure.

Tumor structure 1809 comprises the simulated anatomical structure that defines the structure of a possible facial tumor.

Brain structure 1810 comprises the simulated anatomical structure that defines the brain structure.

Bone structure 1811 comprises the simulated anatomical structure that defines the bone structure.

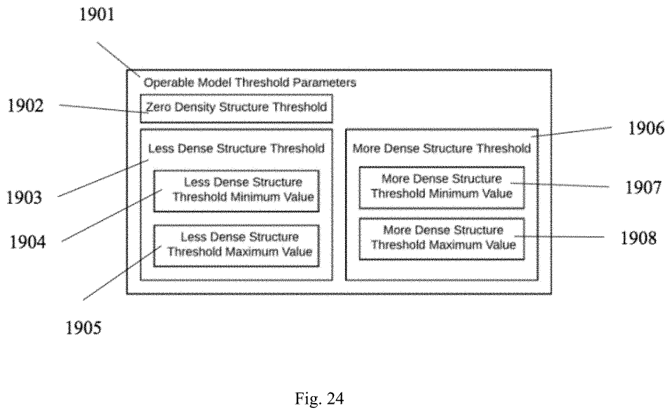

Referring now to FIG. 24, which shows operable model threshold parameters.

Operable model threshold parameters 1901 comprises data or data object which stores information regarding the density thresholds of an anatomical scan. Operable model threshold parameters 1901 preferably comprises more dense structure threshold 1906, less dense structure threshold 1903, and finally zero density structure threshold 1902.

Zero density structure threshold 1902 comprises the range of densities that a person uses to indicate the dead space, or space on the anatomical scan which does not indicate parts of the anatomical model 1802.

Less dense structure threshold 1903 comprises range of density values that a person uses to create a volume for an external structure 601. In some embodiments, it is thought that if less dense structure threshold 1903 is absent then one may be able to hand draw the contours that would allow one to create the mold roi.

Less dense structure threshold 1903 has an alternative embodiment herein termed less dense structure threshold chosen by roi. Less dense structure threshold 1903 comprises less dense structure threshold maximum value 1905 and less dense structure threshold minimum value 1904.

Less dense structure threshold minimum value 1904 comprises data or data object which indicates the range of values in which the minimum less dense structure threshold 1903 can be set to. In some embodiments, less dense structure threshold minimum value 1904 has a preferred density of −200 Hounsfield Units (HU) but in some embodiments, may range from a minimum of −600 Hounsfield Units (HU) to a maximum density of −100 Hounsfield Units (HU).

Less dense structure threshold maximum value 1905 comprises data or data object which stores information regarding the maximum of the range of values in which the less dense structure threshold 1903 can be set to. In some embodiments, less dense structure threshold maximum value 1905 has a preferred density of 150 Hounsfield Units (HU) but in some embodiments, may range from a minimum of 50 Hounsfield Units (HU) to a maximum density of 550 Hounsfield Units (HU).

More dense structure threshold 1906 comprises a range of density values that a person uses to create an internal structure model 901. More dense structure threshold 1906 has an alternative embodiment herein termed more dense threshold chosen by roi. More dense structure threshold 1906 comprises more dense structure threshold minimum value 1907 and more dense structure threshold maximum value 1908.

More dense structure threshold minimum value 1907 comprises a range of values in which the minimum amount of the more dense structure threshold 1906 can be set to. In some embodiments, more dense structure threshold minimum value 1907 has a preferred density of 200 Hounsfield Units (HU) but in some embodiments, may range from a minimum of 200 Hounsfield Units (HU) to a maximum density of 550 Hounsfield.

More dense structure threshold maximum value 1908 comprises a range of values in which the maximum value of the more dense structure threshold 1906 can be set to. In some embodiments, more dense structure threshold maximum value 1908 has a preferred density of 2000 Hounsfield Units (HU) but in some embodiments, may range from a minimum of 200 Hounsfield to a maximum density of 2800 Hounsfield.

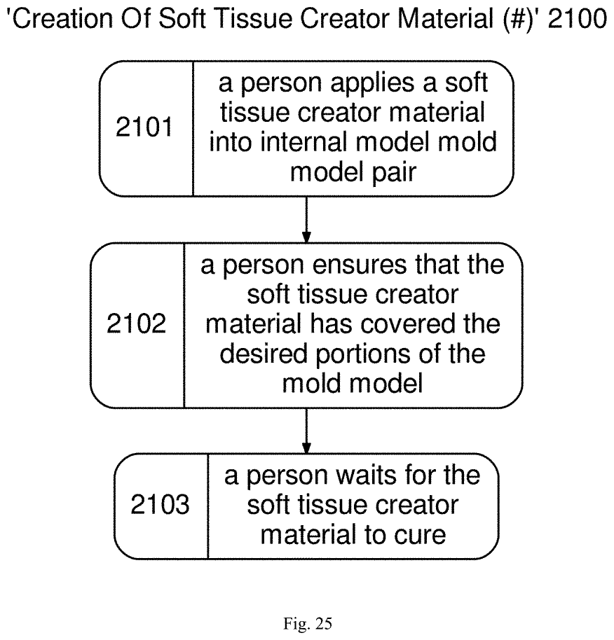

Referring now to FIG. 25, which shows Creation of soft tissue creator material.

In a first step, a person applies a soft tissue creator material 1717 into internal model mold model pair 1501 (Step 2101). Next, a person ensures that the soft tissue creator material 1717 has covered the desired portions of the mold model 1301 (Step 2102). Next, a person waits for the soft tissue creator material 1717 to cure (Step 2103).

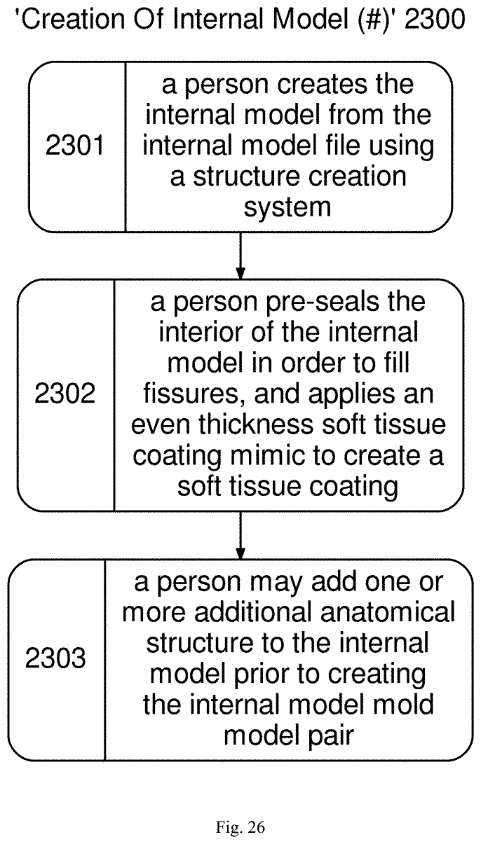

Referring now to FIG. 26, which shows Creation of internal model.

In a first step, a person creates the internal model 1002 from the internal model file 1713 using a structure creation system 1715 (Step 2301). Next, a person pre-seals the interior of the internal model 1002 in order to fill fissures, and applies an even thickness soft tissue coating mimic 1720 to create a soft tissue coating 1719 (Step 2302). Next, a person may add one or more additional anatomical structure to the internal model 1002 prior to creating the internal model mold model pair 1501 (Step 2303).

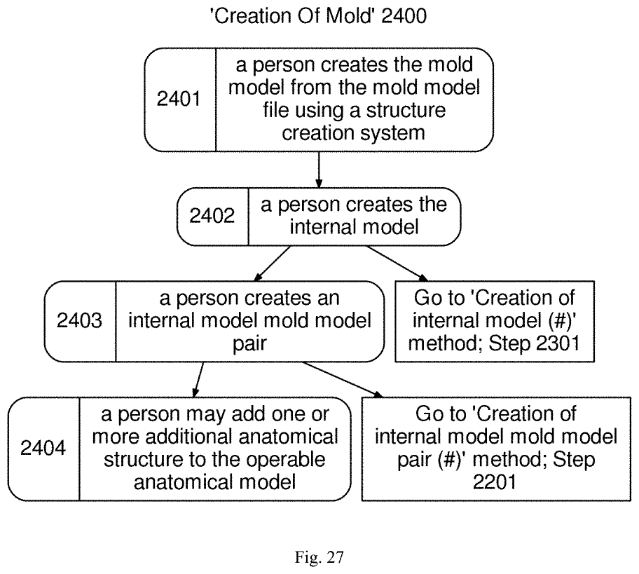

Referring now to FIG. 27, which shows Creation of mold.

In a first step, a person creates the mold model 1301 from the mold model file 1711 using a structure creation system 1715 (Step 2401). Next, a person creates the internal model 1002 (Step 2402). Step 2402 is further detailed below in a related method (2300—‘Creation of internal model (#)’). Next, a person creates an internal model mold model pair 1501 (Step 2403). Step 2403 is further detailed below in a related method (2200—‘Creation of internal model mold model pair (#)’). Next, a person may add one or more additional anatomical structure to the operable anatomical model 501 (Step 2404).

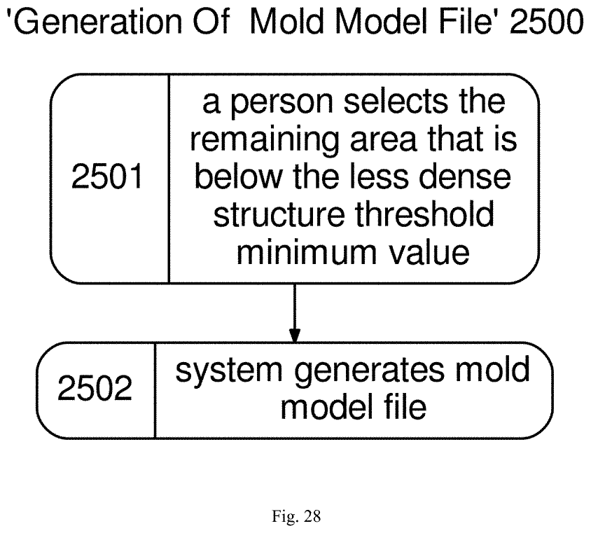

Referring now to FIG. 28, which shows Generation of mold model file.

In a first step, a person selects the remaining area that is below the less dense structure threshold minimum value 1904 (Step 2501). Next, system generates mold model file 1711 (Step 2502).

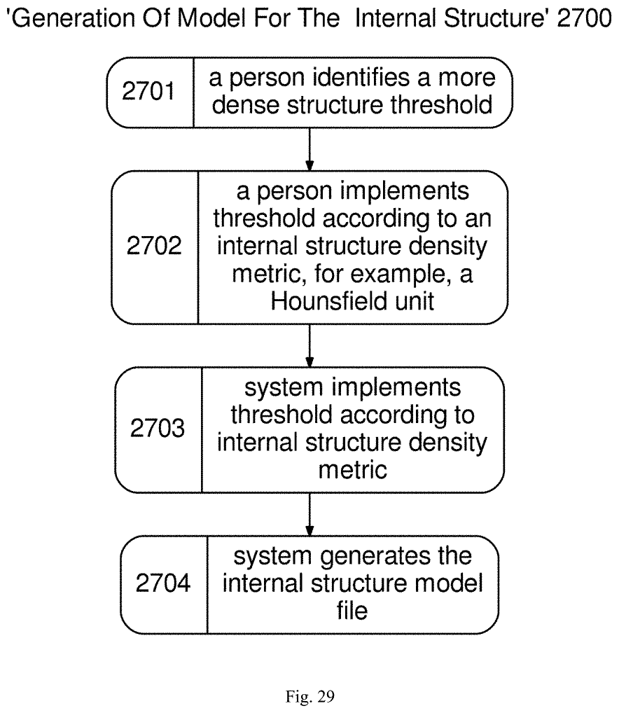

Referring now to FIG. 29, which shows Generation of model for the external structure.

In a first step, a person identifies a less dense structure threshold 1903 (Step 2601). Next, a person implements less dense structure threshold 1903 according to an external structure density metric, for example, a Hounsfield unit (Step 2602). Next, system implements less dense structure threshold 1903 according to an external structure density metric (Step 2603).

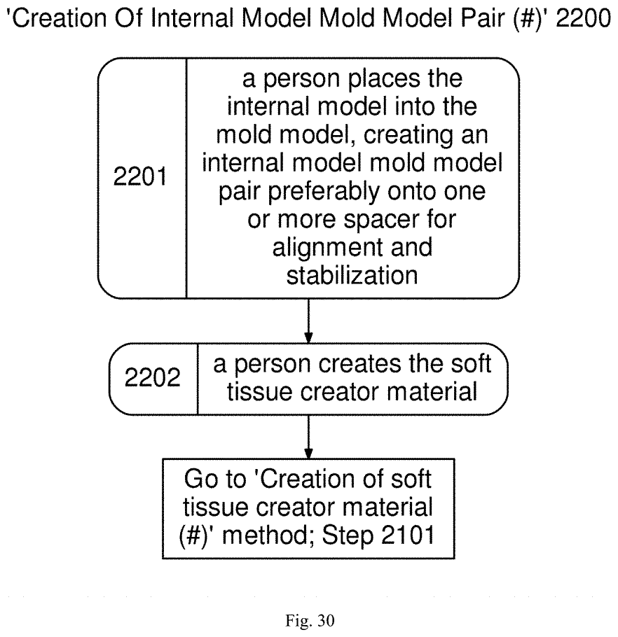

Referring now to FIG. 30, which shows Creation of internal model mold model pair.

In a first step, a person places the internal model 1002 into the mold model 1301, creating an internal model mold model pair 1501 preferably onto one or more spacer 1001 for alignment and stabilization (Step 2201). Next, a person creates the soft tissue creator material 1717 (Step 2202).

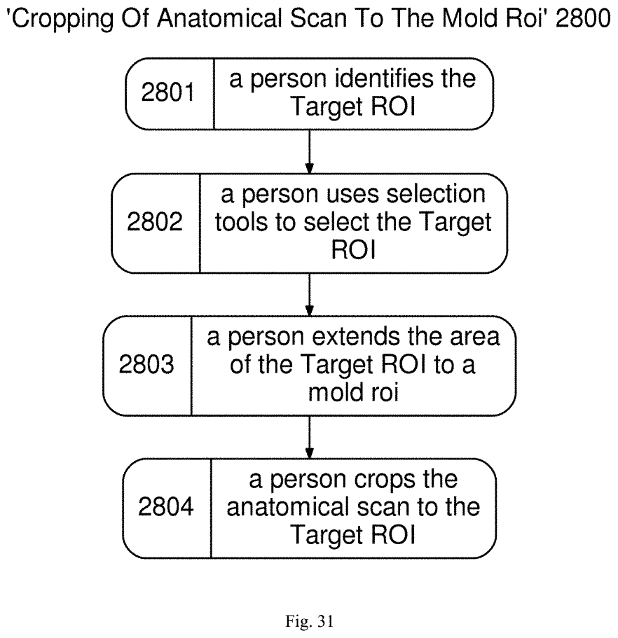

Referring now to FIG. 31, which shows Cropping of anatomical scan to the mold ROI.

In a first step, a person identifies the target roi (Step 2801). Next, a person uses selection tools to select the target roi (Step 2802). Next, a person extends the area of the target roi to a mold ROI (Step 2803). Next, a person crops the target roi to the mold ROI (Step 2804).

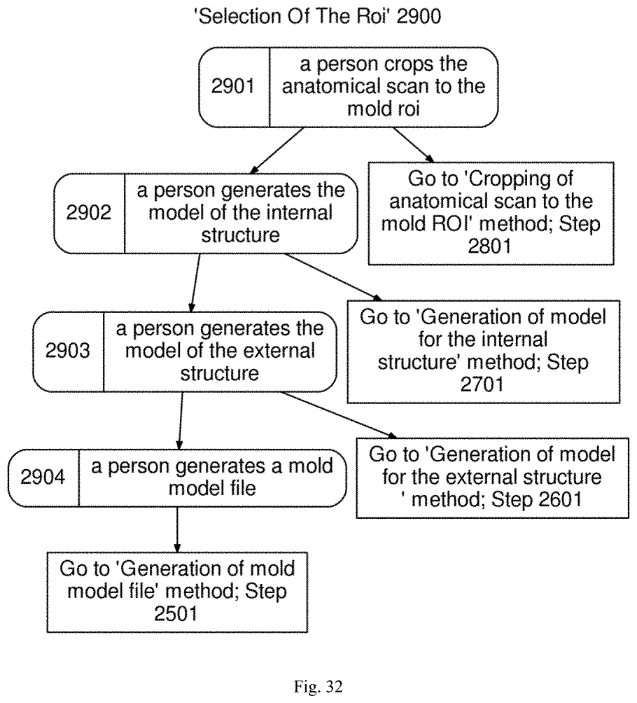

Referring now to FIG. 32, which shows Selection of the ROI.

In a first step, a person crops the anatomical scan to the mold roi (Step 2901). Step 2901 is further detailed below in a related method (2800—‘Cropping of anatomical scan to the mold ROI’). Next, a person generates the model of the internal structure (Step 2902). Step 2902 is further detailed below in a related method (2700—‘Generation of model for the internal structure’). Next, a person generates the model of the external structure 601 (Step 2903). Step 2903 is further detailed below in a related method (2600—‘Generation of model for the external structure’). Next, a person generates a mold model file 1711 (Step 2904). Step 2904 is further detailed below in a related method (2500—‘Generation of mold model file’).

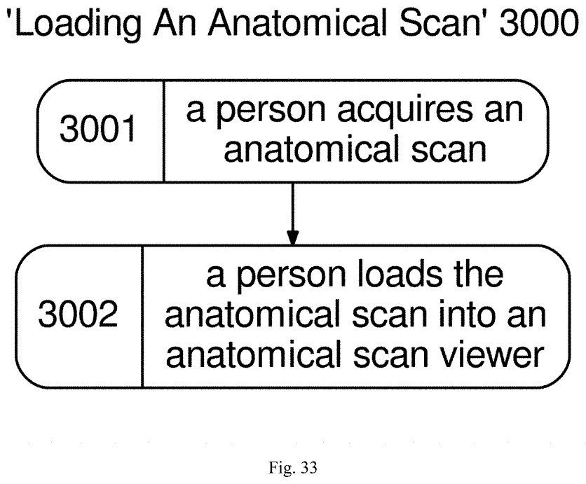

Referring now to FIG. 33, which shows Loading an anatomical scan.

In a first step, a person acquires an anatomical scan (Step 3001). Next, a person loads the anatomical scan into an anatomical scan viewer (Step 3002).

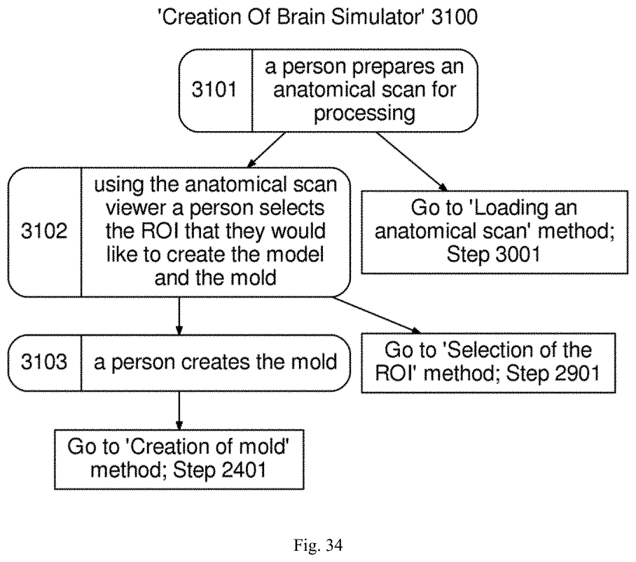

Referring now to FIG. 34, which shows Creation of brain simulator.

In a first step, a person prepares an anatomical scan for processing (Step 3101). Step 3101 is further detailed below in a related method (3000—‘Loading an anatomical scan’). Next, using the anatomical scan viewer a person selects the roi that they would like to create the model and the mold (Step 3102). Step 3102 is further detailed below in a related method (2900—‘Selection of the ROI’). Next, a person creates the mold (Step 3103). Step 3103 is further detailed below in a related method (2400—‘Creation of mold’).

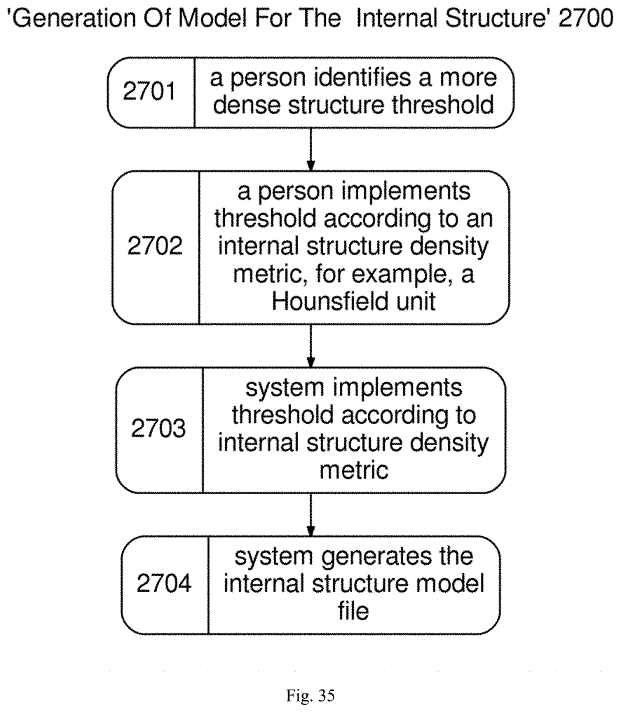

Referring now to FIG. 35, which shows Generation of model for the internal structure.

In a first step, a person identifies a more dense structure threshold 1906 (Step 2701). Next, a person implements threshold according to an internal structure density metric, for example, a Hounsfield unit (Step 2702). Next, system implements threshold according to internal structure density metric (Step 2703). Next, system generates the internal structure model file 1710 (Step 2704).

The following elements and/or terms mold roi, target roi, roi, mold structure, anatomical structure, anatomical scan viewer, anatomical scan, model, mold, more dense threshold chosen by roi, less dense structure threshold chosen by roi, physical ports, independent processor, interface memory, busses, program instructions, system server, system, external structure density metric, internal structure and internal structure density metric are important for the working functionality, but do not appear in the drawings and are shown below.

The region of interest is an acronym which stands for roi.

Anatomical structure comprises a small structural space between tissues or parts of an organ.

Anatomical scan viewer comprises a machine or apparatus for viewing and visually displaying an anatomical scan.

Anatomical scan comprises a computer readable scan of a part of the human anatomy to be modeled by the system. In some embodiments, it is thought that an example of anatomical scan may include CT scan and the like.

Mold comprises encasement in which the operable anatomical model 501 is created.

More dense threshold chosen by roi comprises a version of the more dense structure threshold 1906 in which the maximum and minimum values are set by the roi.

In some embodiments, it is thought that an example of independent processor could be audio processor or perhaps video processor and the like.

In some embodiments, it is thought that an example of interface memory may include volatile and/or non-volatile memory (e.g., RAM) and the like.

Busses comprises a communication system that transfers data between components inside a computer, or between computers.

Program instructions comprises a mechanism for control execution of, or comprise of an operating system, and/or one or more applications. In some embodiments, it is thought that examples of program instructions may include: object code, code produced by a compiler, machine code, code produced by an assembler or a linker, byte code, or code executed using an interpreter.

System server comprises a computing device 107.

External structure density metric comprises a linear transformation of the density of the external structure 601. In some embodiments, it is thought that an example of external structure density metric may include Hounsfield and the like.

Internal structure comprises the less dense and internal structure of an anatomical model 1802.

Internal structure density metric comprises a linear transformation of the density of the internal structure. In some embodiments, it is thought that an example of internal structure density metric may include Hounsfield Unit and the like.

1. An apparatus comprising an operable model file creation system wherein the operable file creation system further comprises:
a. An anatomical scan device;
b. An operable model file;
c. An operable model threshold parameters;
d. A mold creation system; and
e. A structure processing module.

2. The apparatus of claim 1 wherein the structure processing module further comprises:
a. An internal structure processing module;
b. An external structure processing module; and
c. A mold structure processing module.

3. The apparatus of claim 2 wherein the internal structure processing module further comprises:
a. An internal structure threshold processor and;
b. An internal structure file generator.

4. The apparatus of claim 1 wherein the anatomical scan device further comprises:
a. A threshold tool module; and
b. A ROI selection tool module.

5. The apparatus of claim 1 wherein the operable model file further comprises:
a. An internal model file;
b. An external model file;
c. A mold model file; and
d. An internal structure model file.

6. The apparatus of claim 1 wherein the operable model threshold parameters further comprises:
a. A more dense structure threshold;
b. A less dense structure threshold; and
c. A zero density structure threshold.

7. The apparatus of claim 1 wherein the mold creation system further comprises:
a. A structure creation system; and
b. An operable anatomical model.

8. The apparatus of claim 7 wherein the structure creation system further comprises:
a. An internal model;
b. An internal model mold model pair; and
c. A mold model.

9. The apparatus of claim 8 wherein the internal mold model pair further comprises a soft tissue creator model and a soft tissue creator material.

10. The apparatus of claim 8 wherein the mold model comprises a spacer.

11. The apparatus of claim 9 wherein the soft tissue creator model further comprises a soft tissue coating wherein the soft tissue coating further comprise a soft tissue coating mimic.

12. The apparatus of claim 10 wherein there are a minimum of 1 and a maximum of 1—spacers.

13. The apparatus of claim 8 wherein the internal model further comprises:
a. A spacer receiver; and
b. An internal structure model.
